(19) United States
(12) Patent Application Publication
(10) Pub. No.: US 2026/0191515 A1
(43) Pub. Date: Jul. 9, 2026

(54) IN-LINE TISSUE ANALYSIS AND TRANSPORTATION METHOD

(71) Applicant: Devicor Medical Products, Inc., Cincinnati, OH (US)

(72) Inventors: Garrett A. HOUSEHOLDER, Cincinnati, OH (US); Andrew P. NOCK, Dayton, OH (US); Jack A. RANDALL, Cincinnati, OH (US); James O. ROGERS, III, Milford, OH (US)

(21) Appl. No.: 19/556,206

(22) Filed: Mar. 4, 2026

Related U.S. Application Data
(63) Continuation of application No. PCT/US2024/048131, filed on Sep. 24, 2024.
(60) Provisional application No. 63/540,967, filed on Sep. 28, 2023.

(51) Int. Cl. A61B 10/00; A61B 10/02

(52) U.S. Cl. CPC A61B 10/0096; A61B 10/0275

(57) ABSTRACT

A biopsy system includes a biopsy device, a tissue handler, and a tissue transport tube. The biopsy device includes a probe, a needle, and a cutter. The cutter is movable relative to the needle to sever one or more tissue samples. The tissue handler includes a tissue sample holder and an analysis assembly. The tissue sample holder includes a sample tray including a plurality of sample chambers. The sample tray further includes an external surface defining one or more sample stopping features and a plurality of receiving openings. Each receiving opening corresponds to a sample chamber. The tissue transport tube is adapted to connect between the tissue sample holder and the cutter of the biopsy device. The sample tray is configured to move relative to the tissue transport tube to selectively align the one or more sample stopping features or each receiving opening with the tissue transport tube.

### PRIORITY

This application claims priority to U.S. Provisional Application No. 63/540,967, entitled “In-Line Tissue Analysis and Transportation Method,” filed on Sep. 28, 2023, the disclosure of which is hereby incorporated by reference herein.

### BACKGROUND

Biopsy samples have been obtained in a variety of ways in various medical procedures using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, PEM guidance, BSGI guidance, or otherwise.

Merely illustrative biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,432,065, entitled “Method for Using a Surgical Biopsy System with Remote Control for Selecting an Operational Mode,” issued Aug. 13, 2002; U.S. Pat. No. 7,442,171, entitled “Remote Thumbwheel for a Surgical Biopsy Device,” issued Oct. 8, 2008; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued Feb. 21, 2012; U.S. Pat. No. 8,206,316, entitled “Tetherless Biopsy Device with Reusable Portion,” issued Jun. 26, 2012; U.S. Pat. No. 8,491,496, entitled “Biopsy Device with Sample Storage,” issued on Jul. 23, 2013; U.S. Pat. No. 8,702,623, entitled “Biopsy Device with Discrete Tissue Chambers,” issued Apr. 22, 2014; U.S. Pat. No. 8,764,680, entitled “Handheld Biopsy Device with Needle Firing,” issued Jul. 1, 2014; U.S. Pat. No. 9,095,326, entitled “Biopsy System with Vacuum Control Module,” issued Aug. 4, 2015; U.S. Pat. No. 9,326,755, entitled “Biopsy Device Tissue Sample Holder with Bulk Chamber and Pathology Chamber,” issued May 3, 2016; U.S. Pat. No. 9,345,457, entitled “Presentation of Biopsy Sample by Biopsy Device,” issued May 24, 2016; U.S. Pat. No. 10,905,404, entitled “Tissue Sample Holder with Enhanced Features,” issued on Feb. 2, 2021; and U.S. Pat. No. 11,504,101, entitled “Biopsy Device with Remote Multi-Chamber Tissue Sample Holder” issued on Nov. 22, 2022. The disclosure of each of the above-cited U.S. Patents is incorporated by reference herein.

Additional illustrative biopsy devices and biopsy system components are disclosed in U.S. Pat. Pub. No. 2006/0074345, entitled “Biopsy Apparatus and Method,” published Apr. 6, 2006, now abandoned; U.S. Pat. Pub. No. 2010/0152610, entitled “Hand Actuated Tetherless Biopsy Device with Pistol Grip,” published Jun. 17, 2010; U.S. Pat. Pub. No. 2010/0160819, entitled “Biopsy Device with Central Thumbwheel,” published Jun. 24, 2010, now abandoned; U.S. Pat. Pub. No. 2012/0283563, entitled “Biopsy Device with Manifold Alignment Feature and Tissue Sensor,” published Nov. 8, 2012, now abandoned; U.S. Pat. App. No. 2013/0150751, entitled “Biopsy Device With Slide-In Probe,” published Jun. 13, 2013; and U.S. Pat. App. No. 2013/0324882, entitled “Control for Biopsy Device,” published Dec. 5, 2013. The disclosure of each of the above-cited U.S. Patent Application Publications, U.S. Non-Provisional Patent Applications, and U.S. Provisional Patent Applications is incorporated by reference herein.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1A depicts a perspective view of an example biopsy system;

FIG. 1B depicts a detailed perspective view of a needle of a biopsy device of the biopsy system of FIG. 1A;

FIG. 2 depicts a perspective view of an example tissue handling assembly that may be readily incorporated into the system of FIG. 1;

FIG. 3 depicts a perspective view of a tissue sample holder of the tissue handling assembly of FIG. 2;

FIG. 4 depicts a detailed perspective view of a sample tray of the tissue sample holder of FIG. 3;

FIG. 5A depicts another detailed perspective view of the sample tray of FIG. 4, the sample tray being positioned relative to a tissue transport tube to stop a tissue sample;

FIG. 5B depicts yet another detailed elevational view of the sample tray of FIG. 4, the sample tray being positioned relative to the tissue transport tube of FIG. 5A to permit movement of a tissue sample;

FIG. 6 depicts a side elevational view of the tissue handling assembly of FIG. 2 in use to image a tissue sample;

FIG. 7 depicts a perspective view an example alternative tissue sample holder that may be readily incorporated into the tissue handling assembly of FIG. 2;

FIG. 8 depicts a perspective view of another example of an alternative tissue sample holder that may be readily incorporated into the tissue handling assembly of FIG. 2; and

FIG. 9 depicts a top plan view of a sample stopping mechanism of the tissue sample holder of FIG. 8.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### I. Overview of Illustrative Biopsy System

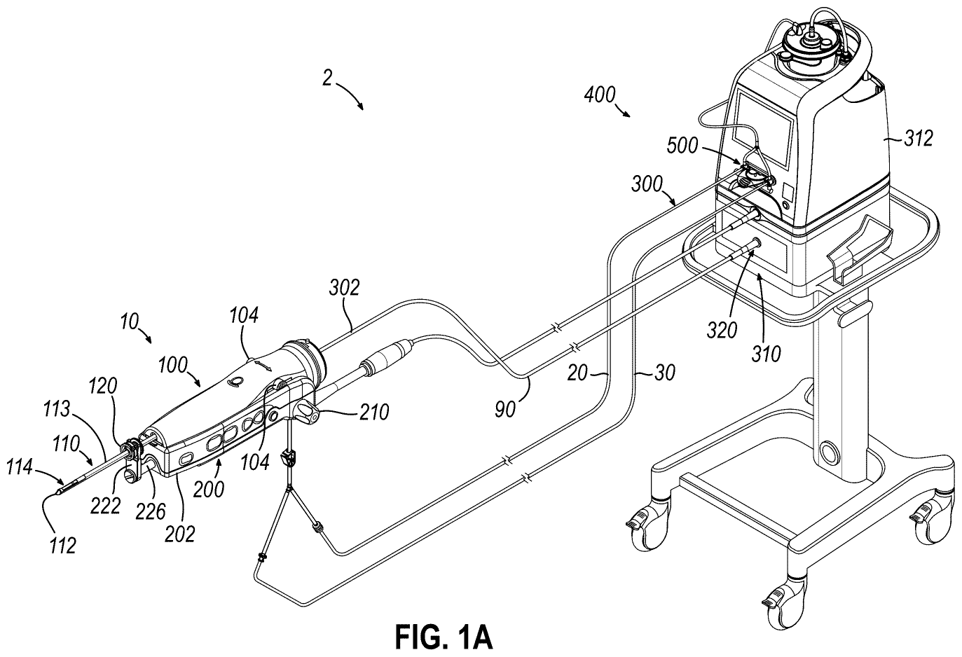

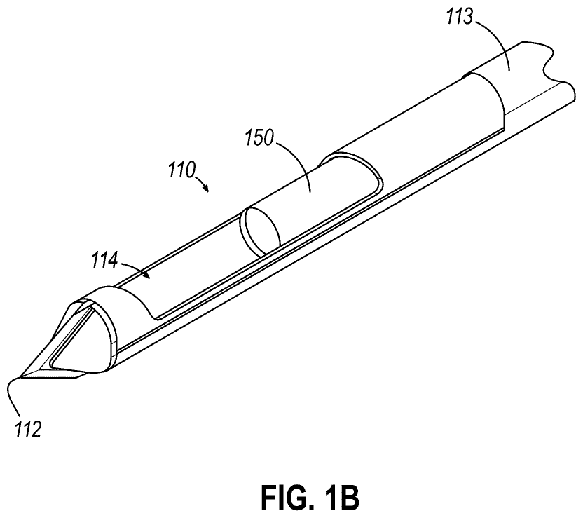

FIG. 1A depicts an illustrative biopsy system (2) including a biopsy device (10) and a vacuum control module (400). Biopsy device (10) of the present example includes a probe (100) and a holster (200). A needle (110) extends distally from probe (100), and is inserted into a patient's tissue to obtain tissue samples. These tissue samples are communicated through needle and into a tissue handling assembly (300) with a tissue transport tube (302) connected at the proximal end of probe (100), as will also be described in greater detail below. It should also be understood that the use of the term “holster” herein should not be read as requiring any portion of probe (100) to be inserted into any portion of holster (200). For instance, in the present example, holster (200) includes a set of prongs (not shown) that are received by at least a portion of probe (100) to releasably secure probe (100) to holster (200). Probe (100) optionally may include one or more resilient tabs (104) that may be pressed inwardly to disengage the prongs, such that a user may simultaneously depress both tabs (104) then pull probe (100) rearwardly and away from holster (200) to decouple probe (100) from holster (200). Of course, a variety of other types of structures, components, features, etc. (e.g., bayonet mounts, latches, clamps, clips, snap fittings, etc.) may be used to provide removable coupling of probe (100) and holster (200). Furthermore, in some biopsy devices (10), probe (100) and holster (200) may be of unitary or integral construction, such that the two components cannot be separated. By way of example only, in versions where probe (100) and holster (200) are provided as separable components, probe (100) may be provided as a disposable component, while holster (200) may be provided as a reusable component. Still other suitable structural and functional relationships between probe (100) and holster (200) will be apparent to those of ordinary skill in the art in view of the teachings herein.

Biopsy device (10) of the present example is configured to mount to a table or fixture, and be used under stereotactic guidance. Of course, biopsy device (10) may instead be used under ultrasound guidance, MRI guidance, PEM guidance, BSGI guidance, or otherwise. It should also be understood that biopsy device (10) may be sized and configured such that biopsy device (10) may be operated by a single hand of a user. In particular, a user may grasp biopsy device (10), insert needle (110) into a patient's breast, and collect one or a plurality of tissue samples from within the patient's breast, all with just using a single hand. Alternatively, a user may grasp biopsy device (10) with more than one hand and/or with any desired assistance. In some settings, the user may capture a plurality of tissue samples with just a single insertion of needle (110) into the patient's breast. Such tissue samples may be pneumatically deposited within at least a portion of tissue handling assembly (300), and later retrieved from tissue handling assembly (300) for further analysis. While examples described herein often refer to the acquisition of biopsy samples from a patient's breast, it should be understood that biopsy device (10) may be used in a variety of other procedures for a variety of other purposes and in a variety of other parts of a patient's anatomy (e.g., prostate, thyroid, etc.). Various example components, features, configurations, and operabilities of biopsy device (10) will be described in greater detail below; while other suitable components, features, configurations, and operabilities will be apparent to those of ordinary skill in the art in view of the teachings herein.

Holster (200) of the present example includes an outer housing (202) that encloses various components that are used to drive various components of probe (100) for the collection of tissue samples. Although not shown, it should be understood that holster (200) of the present example includes one or more gears (not shown) that mesh with corresponding gears of probe (100). In particular, these gears are exposed through an upper portion of outer housing (202) to mesh with corresponding gears of probe (100) when probe (100) and holster (200) are coupled together. This configuration permits holster (200) to communicate rotary motion to probe (100), thereby driving various components of probe (100) for the collection of tissue samples. For instance, the gears may drive an actuation assembly associated with a hollow tubular cutter (150) (see FIG. 1B) within needle (110) to sever tissue samples received within a lateral aperture (114) defined by needle (110). Likewise, other gears may be employed to rotate needle (110).

As noted above, in some examples a gear associated with holster (200) can provide rotation of needle (110) relative to probe (100). In the present example, this rotation is manually initiated by rotating knob (210). In particular, knob (210) is coupled with the gear associated with rotation of needle (110) by a series of gears (not shown) and shafts (not shown), such that rotation of knob (210) rotates needle (110). By way of example only, such a needle rotation mechanism may be constructed in accordance with the teachings of U.S. Pub. No. 2008/0214955, the disclosure of which is incorporated by reference herein. As another merely illustrative example, a needle rotation mechanism may be constructed in accordance with the teachings of U.S. Pub. No. 2010/0160819, the disclosure of which is incorporated by reference herein. In some other versions, needle (110) is rotated by a motor. In still other versions, needle (110) is simply rotated by rotating thumbwheel (116). Various other suitable ways in which rotation of needle (110) may be provided will be apparent to those of ordinary skill in the art in view of the teachings herein. It should also be understood that some versions may provide no rotation of needle (110).

Holster (200) also includes a firing rod (226) and fork (222), which couple with needle (110) and fire needle (110) distally. By way of example only, such firing may be useful in instances where biopsy device (10) is mounted to a stereotactic table fixture or other fixture, with tip (112) adjacent to a patient's breast, such that the needle firing mechanism may be activated to drive needle (110) into the patient's breast. The needle firing mechanism may be configured to drive needle (110) along any suitable range of motion, to drive tip (112) to any suitable distance relative to fixed components of probe (100).

In the present example, the needle firing mechanism is coupled with needle (110) via a firing rod (226) and a firing fork (222). Firing rod (226) and firing fork (222) are unitarily secured together. Firing fork (222) includes a pair of prongs that receive hub member (120) of needle (110) therebetween. The prongs of firing fork (222) are positioned between an annular flange and a thumbwheel of hub member (120), such that needle (110) will translate unitarily with firing rod (226) and fork (222). The prongs nevertheless removably receive hub member (120), such that fork (222) may be readily secured to hub member (120) when probe (100) is coupled with holster (200); and such that hub member (120) may be readily removed from fork (222) when probe (100) is decoupled from holster (200). The prongs are also configured to permit hub member (120) to rotate between the prongs. Other suitable components, configurations, and relationships will be apparent to those of ordinary skill in the art in view of the teachings herein. The internal components of the needle firing mechanism of the present example are configured and arranged as described in U.S. Non-Provisional U.S. Pat. No. 8,858,465, entitled “Biopsy Device with Motorized Needle Firing,” issued on Oct. 14, 2014, the disclosure of which is incorporated by reference herein.

Holster (200) of the present example is powered by one or more motors (not shown) contained within outer housing (202). These motors are generally configured to drive one or more gears to thereby rotate and translate a tubular cutter (150) disposed within needle (110). Holster (200) also includes a motor (not shown) that is operable to drive firing rod (226), to thereby arm and fire needle (110). All motors referred to herein are contained within holster (200) in the present example and receive power from vacuum control module (400) via cable (90). In addition, data may be communicated between vacuum control module (400) and holster (200) via cable (90). In some other versions, one or more motors are powered by one or more batteries located within holster (200) and/or probe (100). It should therefore be understood that, as with other components described herein, cable (90) is merely optional. As yet another merely illustrative variation, motors may be powered pneumatically, such that cable (90) may be substituted with a conduit communicating a pressurized fluid medium to holster (200). As still other merely illustrative variation, cable (90) may include one or more rotary drive cables that are driven by motors that are located external to holster (200). It should also be understood that two or three of the motors may be combined as a single motor. Other suitable ways in which various the motors may be driven will be apparent to those of ordinary skill in the art in view of the teachings herein.

Probe (100) of the present example includes a needle (110) extending distally from probe (100) that is inserted into a patient's tissue to obtain tissue samples. Such tissue samples are transported proximally through needle (110) and into transport tube (302), where the tissue samples may be deposited within at least a portion of tissue handling assembly (300), as will be described in greater detail below. Vacuum control module (400) is coupled with probe (100) via a valve assembly (500) and tubes (20, 30), which is operable to selectively provide vacuum, saline, atmospheric air, and venting to probe (100). Although the present example is shown with a pair of tubes (20, 30), it should be understood that in some examples one tube may be omitted in lieu of a tissue transport tube (302) discussed below. The internal components of the valve assembly of the present example are configured and arranged as described in U.S. Pub. No. 2013/0218047, entitled “Biopsy Device Valve Assembly,” published Aug. 22, 2013, the disclosure of which is incorporated by reference herein.

As described above, probe (100) may include one or more gears to mesh with corresponding gears of holster (200). These gears are operable to drive a cutter actuation mechanism in probe (100). Probe (100) may also include another gear that is configured to mesh with a corresponding gear of holster (200) to thereby rotate needle (110).

Needle (110) of the present example comprises a cannula (113) having a tissue piercing tip (112), a lateral aperture (114) located proximal to tip (112), and a hub member (120). Tissue piercing tip (112) is configured to pierce and penetrate tissue, without requiring a high amount of force, and without requiring an opening to be pre-formed in the tissue prior to insertion of tip (112). Alternatively, tip (112) may be blunt (e.g., rounded, flat, etc.) if desired. By way of example only, tip (112) may be configured in accordance with any of the teachings in U.S. Pat. No. 8,801,742, entitled “Needle Assembly and Blade Assembly for Biopsy Device,” issued on Aug. 12, 2014, the disclosure of which is incorporated by reference herein. As another merely illustrative example, tip (112) may be configured in accordance with at least some of the teachings in U.S. Pat. App. No. 9,486,186, entitled “Biopsy Device with Slide-In Probe,” issued on Nov. 8, 2016, the disclosure of which is incorporated by reference herein. Other suitable configurations that may be used for tip (112) will be apparent to those of ordinary skill in the art in view of the teachings herein.

Lateral aperture (114) is sized to receive prolapsed tissue during operation of device (10). Although not shown, it should be understood that a hollow tubular cutter (150) having a sharp distal edge is located within needle (110). Cutter (150) is operable to rotate and translate relative to needle (110) and past lateral aperture (114) to sever a tissue sample from tissue protruding through lateral aperture (114). For instance, cutter (150) may be moved from an extended position to a retracted position, thereby “opening” lateral aperture (114) to allow tissue to protrude therethrough; then from the retracted position back to the extended position to sever the protruding tissue. As will be described in greater detail below, needle (110) may be rotated to orient lateral aperture (114) at any desired angular position about the longitudinal axis of needle (110). Such rotation of needle (110) is facilitated in the present example by hub member (120), which is described in greater detail below.

In some examples, needle (110) also includes a longitudinal wall (not shown) extending proximally from the proximal portion of tip (112). In such examples, the wall may only extend along a length less than the full length of cannula (113). However, in other examples such a wall may extend the full length of cannula (113) if desired. Where needle (110) includes the wall, the wall may define a two-lumen configuration within needle (110). In examples where the wall only extends for a portion of needle (110), it should be understood that at least a portion of cutter (150) may also define the two-lumen configuration of needle (110). Furthermore, to facilitate fluid flow between lumens, the wall may include a plurality of openings (not shown). An example of such a configuration is disclosed in U.S. Pat. No. 7,918,803, entitled “Methods and Devices for Automated Biopsy and Collection of Soft Tissue,” issued Apr. 5, 2011, the disclosure of which is incorporated by reference herein. Of course, as with any other component described herein, any other suitable configurations may be used.

Hub member (120) of the present example is overmolded about needle (110), such that hub member (120) and needle (110) rotate and translate unitarily with each other. By way of example only, needle (110) may be formed of metal, and hub member (120) may be formed of a plastic material that is overmolded about needle (110) to unitarily secure and form hub member (120) to needle (110). Hub member (120) and needle (110) may alternatively be formed of any other suitable material(s), and may be secured together in any other suitable fashion. In the present example, hub member (120) defines a thumbwheel feature to provide manual rotation of needle. Various other suitable ways in which manual rotation of needle (110) may be provided will be apparent to those of ordinary skill in the art in view of the teachings herein. It should also be understood that rotation of needle (110) may be automated in various ways, including but not limited to the various forms of automatic needle rotation described in various references that are cited herein.

As noted above, needle (110) contains a hollow tubular cutter that is operable to simultaneously translate and rotate relative to needle (110) to sever a tissue sample from tissue protruding through lateral aperture (114). Although not shown, it should be understood that in some examples such a cutter (150) may be operatively coupled to a cutter drive mechanism disposed within probe. Such a cutter drive mechanism may be in communication with one or more gears, which may mesh with one or more corresponding gears of holster (200). The cutter drive mechanism may thus be driven by gears of holster (200) to simultaneously rotate and translate cutter (150) disposed within needle (110). In some examples, cutter (150) drive mechanism may include various threaded and keyed features to facilitate simultaneous rotation and translation of cutter (150). In such examples, a single rotatory input may be converted by the cutter drive mechanism into both rotation and translation of cutter (150). Alternatively, in other examples, rotation and translation of cutter (150) may be supplied by separate rotary inputs. In yet other examples, both rotation and translation of cutter (150) may be provided by two rotary inputs acting together such as by two gears moving at different rotational speeds. In some versions, the foregoing cutter actuation components are further configured in accordance with at least some of the teachings of U.S. Pat. No. 9,345,457, entitled “Presentation of Biopsy Sample by Biopsy Device,” issued on May 24, 2016, the disclosure of which is incorporated by reference herein. As yet another merely illustrative example, cutter (150) disposed within needle (110) may be rotated and/or translated using pneumatic motors, etc. Still other suitable ways in which cutter (150) disposed within needle (110) may be actuated will be apparent to those of ordinary skill in the art in view of the teachings herein.

### II. Illustrative Tissue Handling Assembly

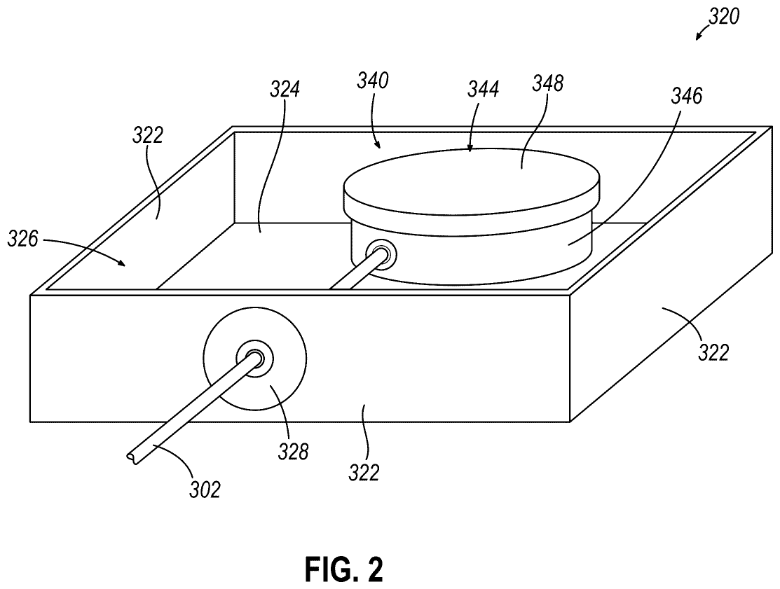

Tissue handling assembly (300) is best seen in FIGS. 1A and 2. As seen in FIG. 1A, at least a portion of tissue handling assembly (300) is incorporated into vacuum control module (400). However, it should be understood that in other examples, tissue handling assembly (300) may be entirely independent and separate from vacuum control module (400). Tissue handling assembly (300) of the present example includes a tissue transport tube (302), and a tissue handler (310). Tissue transport tube (302) extends from tissue handler (310) to biopsy device (10). As will be described in greater detail below, tissue transport tube (302) is generally configured to receive tissue samples from cutter (150) disposed within needle (110) and to communicate such tissue samples from biopsy device (10) to tissue handler (310).

Tissue handler (310) generally includes an outer housing (312) and a collection drawer (320). As will be described in greater detail below, tissue handler (310) is generally configured to receive a plurality of tissue samples from tissue transport tube (302). The tissue samples are then generally arranged in a predetermined configuration. As will also be described in greater detail below, tissue hander (310) may include various sample analysis features to provide procedure room analysis of tissue samples collected within tissue handler (310). Suitable analysis features may include, among other things, x-ray transmitters and receivers, CCD cameras for visual inspection, bioimpedance sensors, and/or etc.

Collection drawer (320) is received within a portion of outer housing (312). Collection drawer (320) is generally translatable into and out of outer housing (312) to provide access to the interior of collection drawer (320) and thereby permit removal of tissue samples and/or various components from tissue handler (310), as will be described in greater detail below. In some examples, collection drawer (320) is manually operable to translate relative to outer housing (312). In other examples, translation of collection drawer (310) is powered by motor driven or pneumatically driven assemblies to provide automatic or semi-automatic translation of collection drawer (310).

As best seen in FIG. 2, the interior of collection drawer (320) includes a plurality of sidewalls (322) and a floor (324) defining an interior space (326). Tissue transport tube (302) is configured to communicate with the interior space (326) of collection drawer (320) through a tube port (328) disposed in at least one sidewall (322) of collection drawer (320). Although not shown, it should be understood that in some examples, sidewalls (322) and/or floor (324) may include one or more ports to provide draining of fluid from collection drawer (320). For instance, in some instances biopsy device (10) is used with saline or other fluid mediums. Through the tissue sample collection process, at least some refuse fluids from the biopsy procedure may flow into interior space (326) of collection drawer (320). Thus, it may be desirable in such examples to include drainage ports through sidewalls (322) and/or floor (324).

In the present example, collection drawer (320) contains a tissue sample holder (340) within interior space (326) defined by sidewalls (322) and floor (324). Tissue sample holder (340) of the present example is generally in communication with tissue transport tube (302) to receive a plurality of tissue samples in a predetermined arrangement or configuration. The exterior of tissue sample holder (340) is defined by a cylindrical or rounded tray container (344) having a cup (346) with a lid (348). Tray container (344) generally houses a sample tray (350). Thus, tray container (344) is configured to removably receive sample tray (350). Tray container (344) is generally desirable to control vacuum applied to sample tray (350) or to manage fluid communicated into one or more portions of sample tray (350). Although the present version includes tray container (344), it should be understood that in other versions, tray container (344) is entirely optional and may be omitted. In such versions, cup (346), lid (348), or both may be omitted.

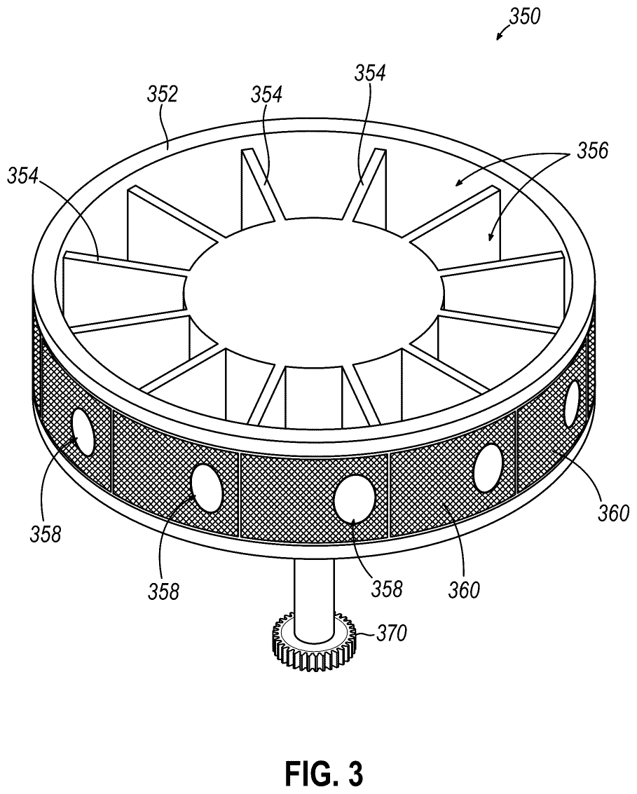

As described above, tissue sample holder (340) of the present example incudes a sample tray (350) (see FIG. 3). Sample tray (350) is configured to receive and house tissue samples in a predetermined arrangement. As will be described in greater detail below, such collected samples may then be imaged or otherwise analyzed via an analysis assembly (380), which is also incorporated into tissue handler (310). As will also be described in greater detail below, sample tray (350) is generally of a rotary configuration such that sample tray (350) or other components associated with sample tray (350) may move within collection drawer (320) to successively collect tissue samples within discrete portions of sample tray (350).

As best seen in FIG. 3, sample tray (350) of the present example includes a cylindrical body (352) with a plurality of divider walls (354) extending outwardly from a central portion of body (352). Divider walls (354) define a plurality of triangular or pie-shaped sample chambers (356) arranged in a circular pattern around the central portion of body (352). Thus, as will be described in greater detail below, sample tray (350) is generally rotatable about the central portion of body (352) to successively received one or more tissue samples within each sample chamber (356).

As will be described in greater detail below, tissue sample holder (340) further includes a driver (370) in communication with sample tray (350). Driver (370) is generally configured to rotate sample tray (350) to move sample chambers (356) about an axis. Although driver (370) is shown in the present example as including a shaft and one or more gears, it should be understood that driver (370) may include other structural features such as motors, cams, additional shafts, additional gears, cables, wheels, and/or etc.

An upper portion of cylindrical body (352) is generally open or otherwise exposed to the exterior of sample tray (350). Thus, each sample chamber (356) is generally accessible from the upper portion of cylindrical body (352). In other examples, the upper portion of rectangular body (352) is closed or otherwise covered relative to the exterior of sample tray (350). In yet other examples, one or more portions of cylindrical body (352) may be configured to be selectively covered such as by a door, cover, casing, and/or etc. Such selective covering may be desirable in some examples to control the flow of vacuum through sample chambers (356), particularly during transport of one or more tissue samples. Thus, in in some examples, each sample chamber (356) may be independently coverable so only the particular sample chamber (356) receiving one or more tissue samples may be covered. In still other examples, such covering may be omitted and vacuum may be controlled by other components such as tray container (344) described above.

A lower portion of cylindrical body (352) includes a floor (not shown). In the present example, the floor is formed of a generally solid material. While the floor of the present example is generally non-permeable, the floor may have at least some permeability in some examples. For instance, in some examples, the floor includes a semi-permeable membrane or a mesh configured to permit fluids to pass through the floor. Such permeability may be desirable to control fluid entering sample tray (350). Thus, in other examples, the floor is generally non-permeable, but may include other fluid management features such as ports, valves, and/or etc. In still other examples, fluid may be controlled through a vacuum tube and/or other structures.

As best seen in FIG. 3, a side portion of cylindrical body (352) defines a plurality of receiving openings (358) in communication with each respective sample chamber (356). Optionally, a portion of cylindrical body (352) such as a lower portion or an interior portion of the central portion of cylindrical body (352) defines one or more vacuum openings (not shown). Such vacuum openings may also be in communication with each respective sample chamber (356) to facilitate the flow of vacuum though a given sample chamber (356). Alternatively, all of sample tray (350) may be placed under vacuum in some examples by vacuum applied to tray container (344).

Each receiving opening (358) is configured to independently communicate with tissue transport tube (302) so that one or more tissue samples may be communicated into a respective sample chamber (356). Correspondingly, in examples including vacuum openings, each vacuum opening is configured to communicate with a vacuum tube (not shown) or other source of vacuum such that vacuum may be communicated into a respective sample chamber (356) to draw one or more tissue samples into the respective sample chamber (356) under vacuum.

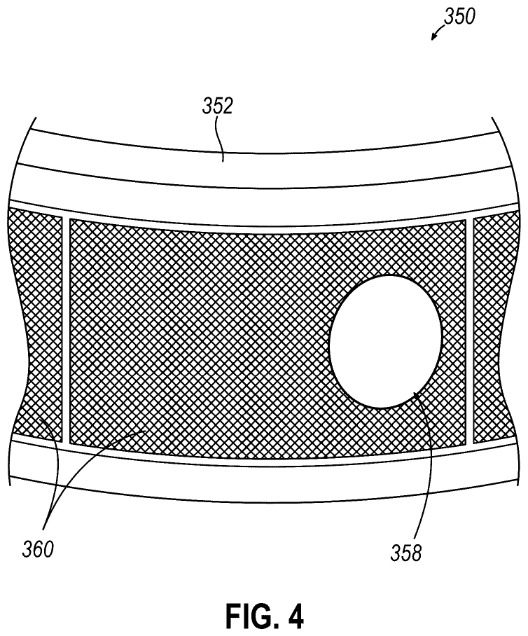

As best seen in FIG. 4, the side portion of cylindrical body (352) further includes a plurality of sample stopping feature (360) proximate each receiving opening (358). Each sample stopping feature (360) is generally configured to block or otherwise stop a tissue sample from passing through cylindrical body (352) and into a corresponding sample chamber (356) while also permitting at least some fluid flow through cylindrical body (352) and into a corresponding sample chamber (356). As will be described in greater detail below, each sample stopping feature (360) is configured to stop a tissue sample within tissue transport tube (302) for imaging or other analysis before transporting the stopped tissue sample into a sample chamber (356) via a corresponding receiving opening (358).

In the present version, each sample chamber (356) is associated with a corresponding sample stopping feature (360). Thus, each sample chamber (356) is in communication with a corresponding sample stopping feature (360) and receiving opening (358). In this configuration, a given receiving opening (358) is positioned proximate one side of a corresponding sample chamber (356), while a given sample stopping feature (360) is positioned proximate an opposite side of the corresponding sample chamber (356). Thus, receiving openings (356) and sample stopping features (360) are generally positioned along a line and are generally proximate each other. This configuration may be desirable to permit rotation of sample tray (350) to index tissue transport tube (302) between receiving openings (356) and sample stopping features (360). Although the present example is shown as each sample stopping feature (360) defining a respective receiving opening (358), sample stopping feature (360) and each respective receiving opening (358) may be separated from each other in other examples.

Each sample stopping feature (360) of the present example is configured as a porous structure integrated into a portion of cylindrical body (352). Suitable porous structures may include a variety of configurations. For instance, in the present example, a mesh or screen configuration is used. In other examples, a plurality of discrete holes, bores, or openings are used. In yet other examples, certain semi-permeable membranes are used. In still other examples, such porous structures are omitted entirely and movable gate structures are used in lieu of porous structures. Although sample stopping features (360) in the present example are integral with cylindrical body (352), it should be understood that in other examples, sample stopping features (360) may be separate from cylindrical body (352) and fixedly or removably attached thereto.

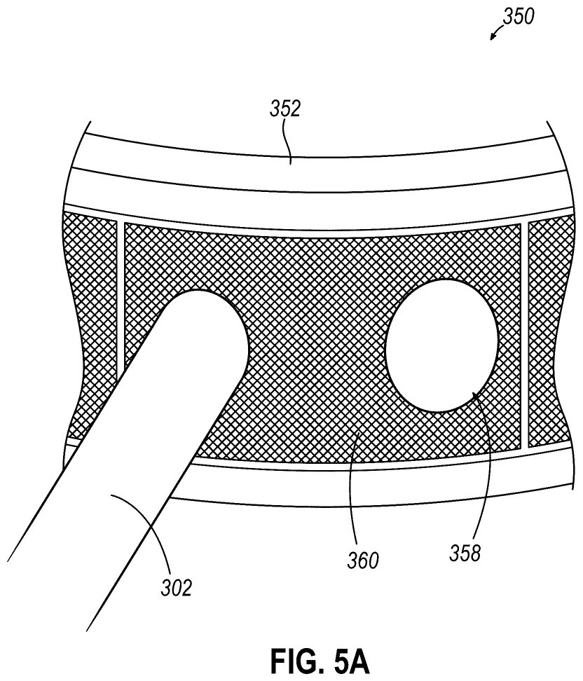

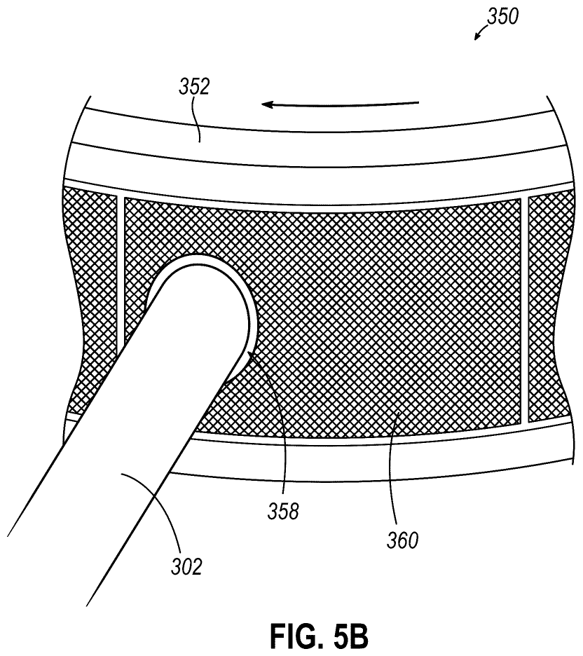

As best seen in FIGS. 5A and 5B, sample tray (350) is disposed at a proximal end of tissue transport tube (302). As will be described in greater detail below, sample tray (350) is generally rotatable relative to tissue transport tube (302) via driver (370) to move tissue transport tube (302) between a sample stopping configuration and a sample acquisition configuration for each sample chamber (356). Tissue transport tube (302) is generally aligned along a sampling axis extending generally perpendicular relative to a rotation axis of sample tray (350). In some examples, a vacuum tube (not shown) may likewise be aligned with the sampling axis or is alternatively aligned along the rotation axis. In examples including a vacuum tube, tissue transport tube (302) and the vacuum tube are each configured to simultaneously be in communication with a selected sample chamber (356) of the plurality of sample chambers (356) depending on the rotational position of sample tray (350) relative to tissue transport tube (302).

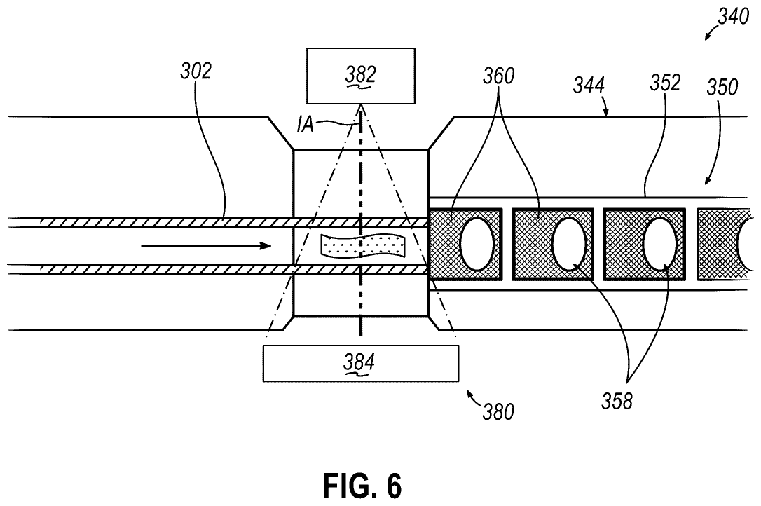

As best seen in FIG. 6, tissue sample holder (340) is proximate analysis assembly (380) to facilitate imaging and/or analysis of tissue samples before being communicated into sample tray (350). In particular, analysis assembly (380) includes one or more imaging elements configured to image or otherwise analyze tissue samples contained within tissue transport tube (302). In the present example, analysis assembly (380) is configured for x-ray imaging. Thus, analysis assembly (380) includes a source (382) and a detector (384) oriented on opposing sides of tissue transport tube (302) to define an imaging axis (IA). In the present example, detector (384) may be integrated into a portion of tissue handler (310) such as collection drawer (320) or another component such as an outer housing. Meanwhile, source (382) is oriented above detector (384) and may be integrated into another portion of tissue handler (310) such as an upper housing. Both detector (384) and source (382) are generally centered about tissue transport tube (302) so that a tissue sample may be imaged prior to being communicated into a sample chamber (356) of sample tray (350).

As best seen in FIGS. 5A through 6, in use, sample tray (350) is configured to receive tissue samples in each sample chamber (356), with analysis assembly (380) being configured to image or otherwise analyze each tissue sample within tissue transport tube (302) before communication to a given sample chamber (356). As shown in FIG. 5A, sample tray (350) is initially rotated by driver (370) into a sample stopping position. In this position, sample tray (350) is positioned to align a sample stopping feature (360) with the sampling axis defined by tissue transport tube (302). When a given sample stopping feature (360) is aligned with tissue transport tube (302), vacuum may be communicated through sample stopping feature (360) to facilitate tissue transport through tissue transport tube (302). Additionally, sample stopping feature (360) may block progression of a tissue sample within tissue transport tube (302) into an associated sample chamber (356).

When a tissue sample is blocked by sample stopping feature (360), the blocked tissue sample may be imaged or otherwise analyzed by analysis assembly (380). As best seen in FIG. 6, source (382) and detector (384) may operative cooperatively to image or otherwise analyze the blocked tissue sample along imaging axis (IA) while the blocked tissue sample remains within tissue transport tube (302).

After imaging and/or analysis of the blocked tissue sample, sample tray (350) may be rotated from the sample stopping position shown in FIG. 5A to a sample acquisition position shown in FIG. 5B. In this position, sample tray (350) is moved to position a receiving opening (358) of a given sample chamber (356) into alignment with the sampling axis defined by tissue transport tube (302). The subsequently blocked tissue sample is then free to pass though receiving opening (358), out of tissue transport tube (302), and into the given sample chamber (356).

Once the subsequently blocked tissue sample is received within a given sample chamber (356), the same rotational process described above can be repeated to both image and/or analyze and capture one or more additional tissue samples. This process may be repeated until each sample chamber (356) is filled or a desired number of tissue samples have been collected. In the present example, capturing of tissue samples is performed successively so that each successive sample chamber (356) is filled. However, in other examples, different sequences may be used. For instance, in some examples, every other sample chamber (356) may be filled. In addition, or in the alternative, rotation may be reversed in some examples to capture multiple samples in a single sample chamber (356).

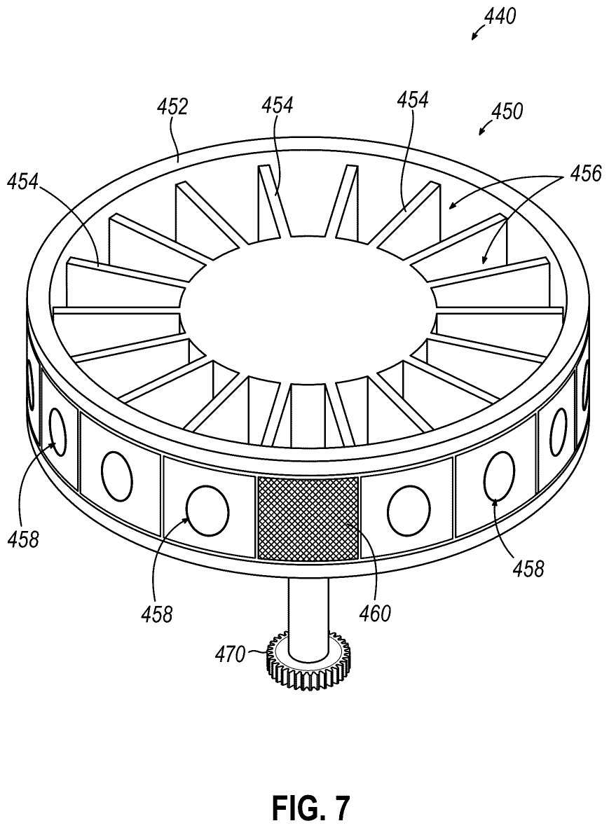

FIG. 7 shows an illustrative alternative tissue sample holder (440) that may be readily incorporated into tissue handler (310) described above either in-lieu of, or in addition to, tissue sample holder (340). As with tissue sample holder (340) described above, tissue sample holder (440) of the present example includes a sample tray (450) and a driver (470). Driver (470) of the present example is substantially similar to driver (370) described above. For instance, driver (470) is generally configured to rotate sample tray (450) and may include various structural features such as shafts, gears, motors, cams, cables, wheels, and/or etc.

As with sample tray (350) described above, sample tray (450) of the present example includes a cylindrical body (452) with a plurality of divider walls (454) extending outwardly from a central portion of body (452). Divider walls (454) define a plurality of triangular or pie-shaped sample chambers (456) arranged in a circular pattern around the central portion of body (452). Thus, as will be described in greater detail below, sample tray (450) is generally rotatable about the central portion of body (452) to successively receive one or more tissue samples within each sample chamber (456).

Also like sample tray (350) described above, a side portion of cylindrical body (452) defines a plurality of receiving openings (458) in communication with each respective sample chamber (456). Optionally, a portion of cylindrical body (452) such as a lower portion or an interior portion of the central portion of cylindrical body (452) defines one or more vacuum openings (not shown). Such vacuum openings may also be in communication with each respective sample chamber (456) to facilitate the flow of vacuum though a given sample chamber (456). Alternatively, all of sample tray (450) may be placed under vacuum in some examples by vacuum applied to tray container (344).

Each receiving opening (458) is configured to independently communicate with tissue transport tube (302) so that one or more tissue samples may be communicated into a respective sample chamber (456). Correspondingly, in examples including vacuum openings, each vacuum opening is configured to communicate with a vacuum tube (not shown) or other source of vacuum such that vacuum may be communicated into a respective sample chamber (456) to draw one or more tissue samples into the respective sample chamber (456) under vacuum.

Like sample tray (350) described above, the side portion of cylindrical body (452) further includes a sample stopping feature (460). However, unlike sample tray (350) described above, sample tray (450) of the present example only includes a single sample stopping feature (460). As will be described in greater detail below, the single sample stopping feature (460) configuration of the present example may be desirable to permit sample tray (450) to have one or more additional sample chambers (456) relative to sample chambers (356) described above within the same footprint. In other words, single sample stopping feature (460) is configured to act as a sample stopping feature (460) for all of sample chambers (456) rather than having a dedicated sample stopping feature for each sample chamber (456). As a result, less total area is occupied by sample stopping feature (460).

Like sample stopping features (360) described above, sample stopping feature (460) of the present example is generally configured to block or otherwise stop a tissue sample from passing through cylindrical body (352) and into the interior of body (352) while also permitting at least some fluid flow through cylindrical body (352). As will be described in greater detail below, sample stopping feature (460) is configured to stop a tissue sample within tissue transport tube (302) for imaging or other analysis before transporting the stopped tissue sample into a sample chamber (456) via a given receiving opening (458).

In the present version, only a single sample chamber (456) is associated with sample stopping feature (460). As a result, the sample chamber (456) corresponding to sample stopping feature (460) may be characterized as an un-occupiable sample chamber (456). In other words, the sample chamber (456) associated with sample stopping feature (460) is unable to receive tissue samples because sample stopping feature (460) defines no opening or port for communication of tissue samples. In some examples, this configuration may be characterized as sample stopping feature (460) being associated with a void defined by sample tray (450) or a dead chamber. Although the particular sample chamber (456) associated with sample stopping feature (460) is shown as having a similar shape and/or size relative to the other sample chambers (456), this sample chamber (456) may have a different shape, size, and/or other characteristics in some examples. In other examples, sample stopping feature (460) may define or be associated with a receiving opening similar to receiving openings (458) to permit sample stopping feature (460) to both block tissue samples and receive tissue samples similarly to the combination of sample stopping features (360) and receiving openings (358) described above.

Sample stopping feature (460) of the present example is configured as a porous structure integrated into a portion of cylindrical body (452). Suitable porous structures may include a variety of configurations. For instance, in the present example, a mesh or screen configuration is used. In other examples, a plurality of discrete holes, bores, or openings are used. In yet other examples, certain semi-permeable membranes are used. In still other examples, such porous structures are omitted entirely and movable gate structures are used in lieu of porous structures. Although sample stopping feature (460) in the present example is integral with cylindrical body (452), it should be understood that in other examples, sample stopping feature (460) may be separate from cylindrical body (452) and fixedly or removably attached thereto.

In use, tissue sample holder (440) is used similarly to tissue sample holder (340) described above. For instance, as similarly described above, sample tray (440) is rotated using driver (470) to align various portions of sample tray (440) with the sampling axis defined by tissue transport tube (302) to block a tissue sample for imaging and/or analysis purposes and then receive the subsequently blocked tissue sample for sample acquisition within each sample chamber (456).

However, unlike the use described above with respect to sample tray (350), the present use of sample tray (450) includes a different sequence of rotation to account for the single sample stopping feature (460) configuration of the present example. For instance, sample tray (450) is first rotated to align sample stopping feature (460) with the sampling axis defined by tissue transport tube (302) to block a tissue sample and hold the blocked tissue example within tissue transport tube (302) for imaging and/or analysis purposes. After imaging and/or analysis, sample tray (450) is rotated to align a selected receiving opening (458) with the sampling axis defined by tissue transport tube (302) and the subsequently blocked tissue sample can be transported into a given sample chamber (456) to capture the tissue sample. After the tissue sample is captured, sample tray (450) is then rotated to again align sample stopping feature (460) with the sampling axis. A subsequent tissue sample can then be imaged and/or analyzed and captured using the same process. The process can be repeated for each receiving opening (458) and sample chamber (456) combination until each sample chamber (456) is filled or a desired number of tissue samples have been acquired.

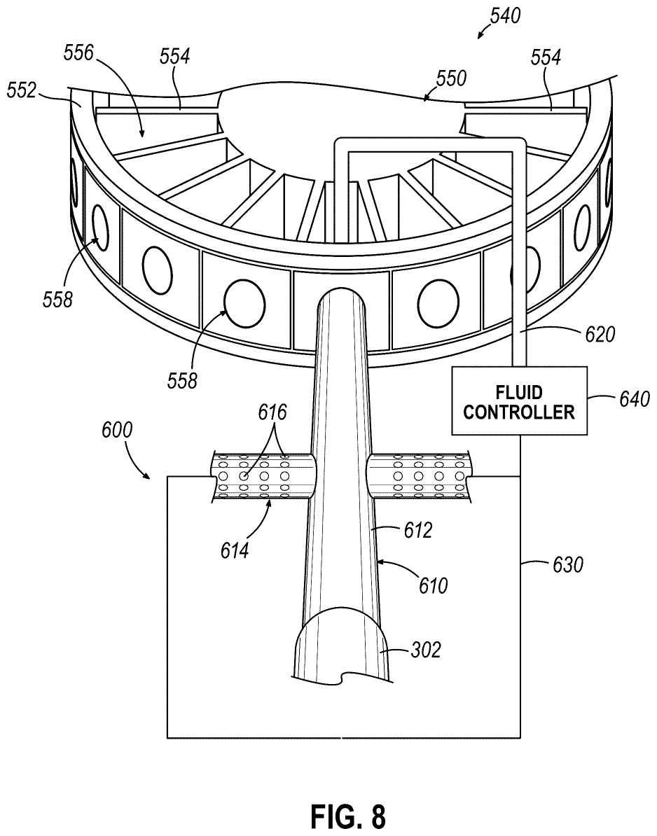

FIG. 8 shows an illustrative alternative tissue sample holder (540) that may be readily incorporated into tissue handler (310) described above either in-lieu of, or in addition to, tissue sample holders (340, 440). Tissue sample holder (540) of the present example is substantially similar to tissue sample holder (440) described above. For instance, as with tissue sample holder (440) described above, tissue sample holder (540) of the present example includes a sample tray (550) and a driver (not shown). The driver of the present example is substantially similar to driver (470) described above such that the driver is generally configured to rotate sample tray (550) and may include various structural features such as shafts, gears, motors, cams, cables, wheels, and/or etc.

As with sample tray (450) described above, sample tray (550) of the present example includes a cylindrical body (552) with a plurality of divider walls (554) extending outwardly from a central portion of body (552). Divider walls (554) define a plurality of triangular or pie-shaped sample chambers (556) arranged in a circular pattern around the central portion of body (552). Thus, as will be described in greater detail below, sample tray (550) is generally rotatable about the central portion of body (552) to successively receive one or more tissue samples within each sample chamber (556).

Also like sample tray (450) described above, a side portion of cylindrical body (552) defines a plurality of receiving openings (558) in communication with each respective sample chamber (556). Optionally, a portion of cylindrical body (552) such as a lower portion or an interior portion of the central portion of cylindrical body (552) defines one or more vacuum openings (not shown). Such vacuum openings may also be in communication with each respective sample chamber (556) to facilitate the flow of vacuum though a given sample chamber (556). Alternatively, all of sample tray (550) may be placed under vacuum in some examples by vacuum applied to tray container (344).

As with receiving openings (458) described above, each receiving opening (558) is configured to independently communicate with tissue transport tube (302) so that one or more tissue samples may be communicated into a respective sample chamber (556). Correspondingly, each receiving opening (558) is configured to communicate with a vacuum tube (620) or other source of vacuum such that vacuum may be communicated into a respective sample chamber (556) to draw one or more tissue samples into the respective sample chamber (556) under vacuum.

Unlike sample tray (450) described above, the side portion of cylindrical body (552) omits structures similar to sample stopping feature (460). Although it should be understood that in some examples, such structures may optionally be included. In addition to or in lieu of structures such as sample stopping feature (460), tissue sample holder (540) of the present example is equipped with or associated with a sample stopping assembly (600). Generally, sample stopping assembly (600) is configured to manipulate vacuum applied to a tissue sample to stop the tissue sample within sample stopping assembly (600) for imaging purposes outside of sample tray (450). Sample stopping assembly (600) may be preferable to structure similar to sample stopping feature (460) in some circumstances. For instance, in some circumstances, examples including sample stopping feature (460) may provide limited spacing between a stopped tissue sample and other structures of tissue sample holder (440, 540), which may lead to interference with imaging of the tissue sample, depending on particular imaging elements used. By contrast, aspects of sample stopping assembly (600) may provide at least some predetermined separation between the stopped tissue sample and other elements of tissue sample holder (540), thereby providing unobstructed imaging in some circumstances.

Sample stopping assembly (600) includes a sample tube (610), a transport vacuum tube (620), vacuum diversion tubes (630), and a fluid controller (640). Sample tube (610) is generally in communication with fluid controller (640) to selectively control the progress of a tissue sample through sample tube (610) as will be described in greater detail below.

Sample tube (610) includes a sample receiving tube (612) and two stopping ports (614) extending perpendicularly relative to sample receiving tube (612). Sample receiving tube (612) is sized and configured to receive a tissue sample. Additionally, sample receiving tube (612) is configured for imaging of a tissue sample. Thus, sample receiving tube (612) may be optically transparent, radiolucent, and/or etc., depending on the particular imaging modality being used. Sample receiving tube (612) extends distally from sample tray (550) toward tissue transport tube (302), such that a distal end of sample receiving tube (612) may be in communication with tissue transport tube (302). Thus, sample receiving tube (612) is hollow and is configured to receive tissue samples from tissue transport tube (302).

Stopping ports (614) extend perpendicularly from sample tube (610) relative to a longitudinal axis defined by sample tube (610). Stopping ports (614) additionally extend from each other. Stopping ports (614) are optionally positioned along a common axis. As will be understood, such positioning along a common axis may be desirable in some examples to balance fluid pressure with respect to a tissue sample. Each stopping port (614) is generally in communication with the hollow interior of sample tube (610) to communicate fluids between stopping ports (614) and sample tube (610).

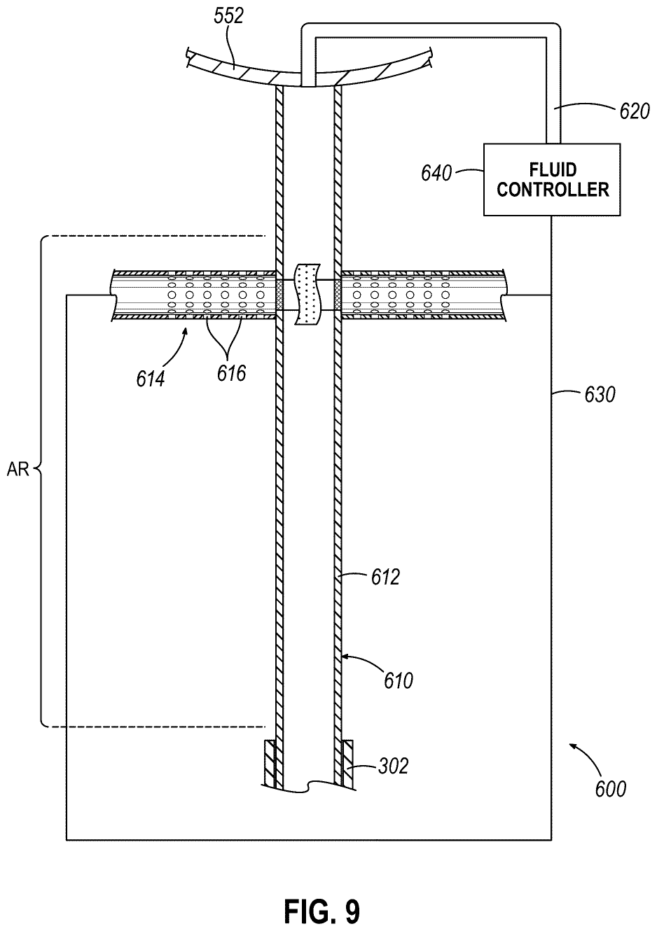

As best seen in FIG. 9, each stopping port (614) includes a plurality of perforations (616) (also collectively referred to as a porous portion in some examples) extending through the surface of each stopping port (614). As will be described in greater detail below, perforations (616) are generally collectively configured as a porous structure to permit the flow of fluid through the wall defining each stopping port (614), while preventing the flow of solid materials such as tissue. In the present example, perforations (616) are oriented perpendicularly relative to the longitudinal axis defined by sample tube (610). In some examples, perforations (616) may be configured as a plurality of thin slots oriented perpendicularly relative to the longitudinal axis defined by sample tube (610). In other examples, perforations (616) may be configured as a plurality of openings or bores arranged in a plurality of linear formations. Of course, other suitable configurations for perforations (616) will be apparent to those of ordinary skill in the art in view of the teachings herein.

Sample tube (610) is in communication with fluid controller (640) via transport vacuum tube (620) and vacuum diversion tubes (630). In particular, transport vacuum tube (620) is in communication with sample tray (550), which is in fluid communication with the proximal end of sample tube (610) via a given receiving opening (558) associated with an indexed sample chamber (556). Although not shown, it should be understood that tissue sample holder (540) may include other features associated with transport vacuum tube (620) to direct fluid communication into sample tray (550) such as covers, manifolds, seals, and/or etc. Regardless, it should be understood the proximal end of sample tube (610) is generally in communication with transport vacuum tube (620) to communicate with fluid controller (640).

Meanwhile, stopping ports (614) are in communication with vacuum diversion tubes (630) such that stopping ports (614) are also in communication with fluid controller (640). In this configuration, fluid controller (640) is configured to control the flow of vacuum or other fluid to the proximal end of sample tube (610) or stopping ports (614) to influence movement of a tissue sample through sample tube (610). In particular, fluid controller (640) may apply vacuum to the proximal end of sample tube (610) to move a tissue sample axially through tissue transport tube (302) and/or sample tube (610). Similarly, fluid controller (640) may apply vacuum to stopping ports (614) to stop axial progress of a tissue sample through sample tube (610).

Fluid controller (640) may be in a variety of forms configured to selectively control the flow of fluid to transport vacuum tube (620) and/or vacuum diversion tubes (630). In some examples, fluid controller (640) may include a vacuum manifold configured to route the communication of fluid therethrough. In other examples, fluid controller (640) may include a stop cock or other valve. Regardless, in such examples, fluid controller (640) may be actuated manually or electronically using a control system.

In use, fluid controller (640) may first be configured to communicate a vacuum to the proximal end of sample tube (610) via transport vacuum tube (620). With sample tube (610) under vacuum, a tissue sample may be communicated through tissue transport tube (302) and into sample tube (610). Once the tissue sample is received within sample tube (610), fluid controller (640) may next communicate vacuum to stopping ports (614) via vacuum diversion tubes (630). This will shift the flow of vacuum from being axial with sample tube (610) to perpendicular, thereby stopping the tissue sample within sample tube (610). However, vacuum may continue to flow via perforations (616), thereby permitting the evacuation of fluids from sample tube (610) while other solid materials such as the tissue sample remain in position.

While the tissue sample is stopped within sample tube (610) imaging may be performed. FIG. 9 illustrates an imaging area (AR) where imaging of the tissue sample may occur. As can be seen, imaging area (AR) is offset relative to sample tray (550), thereby providing imaging unobstructed by elements of sample tray (550).

After imaging, the tissue sample may be communicated into sample tray (550) via sample tube (610). In particular, fluid controller (640) may next shift to communicating vacuum back to the proximal end of sample tube (610) via transport vacuum tube (620). With vacuum applied to the proximal end of sample tube (610), the tissue sample may be pulled proximally though sample tube (610) and into a sample chamber (556) of sample tray (550). The process may then be repeated to collect and image any number of subsequent tissue samples.

In some examples, actuation of fluid controller (640) may be associated with rotation of sample tray (550). For instance, fluid controller (640) may be responsive to rotation of sample tray (550) to shift communication of vacuum to the proximal end of sample tube (610) after sample tray (550) rotates to index sample tube (610) from one sample chamber (556) to another sample chamber (556). This may be beneficial as such indexing may be indicative of sample tray (550) being positioned for receipt of a new tissue sample, thus correlating with axial transportation of a tissue sample through sample tube (610). In other examples, other suitable features may be associated with actuation of fluid controller (640) such as operating of imaging elements, operation of biopsy device (10), and/or etc. In addition, or in the alternative, actuation of fluid controller (640) may be at least partially controlled by timers, sensors, and/or user inputs.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A biopsy system, the biopsy system comprising: a biopsy device, the biopsy device including: a probe, a needle extending from the probe, and a cutter, the cutter being movable relative to the needle to sever one or more tissue samples; tissue handler in communication with the biopsy device, the tissue handler including a tissue sample holder and an analysis assembly, the tissue sample holder including a sample tray, the sample tray including a plurality of sample chambers configured to receive the one or more tissue samples severed by the cutter, the sample tray further including an external surface defining one or more sample stopping features and a plurality of receiving openings, each receiving opening corresponding to a sample chamber; and a tissue transport tube adapted to connect between the tissue sample holder and the cutter of the biopsy device, the sample tray being configured to move relative to the tissue transport tube to selectively align the one or more sample stopping features or each receiving opening with the tissue transport tube.

### EXAMPLE 2

The biopsy system of Example 1, each sample stopping feature of the one or more sample stopping features being porous.

### EXAMPLE 3

The biopsy system of Examples 1 or 2, each sample stopping feature of the one or more sample stopping features defining a mesh configuration.

### EXAMPLE 4

The biopsy system of any of Examples 1 through 3, the one or more sample stopping features including a single sample stopping feature.

### EXAMPLE 5

The biopsy system of any of Examples 1 through 3, the one or more sample stopping features including a single sample stopping feature, the single sample stopping feature being proximate a void defined by the sample tray.

### EXAMPLE 6

The biopsy system of any of Examples 1 through 3, the one or more sample stopping features including a plurality of sample stopping features, each sample stopping feature corresponding to a sample chamber of the plurality of sample chambers.

### EXAMPLE 7

The biopsy system of any of Examples 1 through 3, the one or more sample stopping features including a plurality of sample stopping features, each sample stopping feature corresponding to a sample chamber of the plurality of sample chambers, each sample stopping feature being disposed proximate a respective receiving opening.

### EXAMPLE 8

The biopsy system of any of Examples 1 through 3, the one or more sample stopping features including a plurality of sample stopping features, each sample stopping feature corresponding to a sample chamber of the plurality of sample chambers, each sample stopping feature defining a respective receiving opening.

### EXAMPLE 9

The biopsy system of any of Examples 1 through 8, the sample tray being configured to rotate relative to the tissue transport tube.

### EXAMPLE 10

The biopsy system of any of Examples 1 through 8, the sample tray being configured to rotate relative to the tissue transport tube about an axis of rotation, each receiving opening and sample stopping feature being aligned along a plane oriented perpendicularly relative to the axis of rotation.

### EXAMPLE 11

The biopsy system of any of Examples 1 through 10, the tissue sample holder further including a driver, the driver being configured to rotate the sample tray about an axis.

### EXAMPLE 12

The biopsy system of any of Examples 1 through 10, the tissue sample holder further including a driver, the driver being configured to rotate the sample tray about a rotation drive axis, the plurality of sample chambers being aligned along a plane oriented perpendicularly relative to the rotation drive axis.

### EXAMPLE 13

The biopsy system of any of Examples 1 through 12, the analysis assembly including an x-ray source and an x-ray detector, the x-ray source and the x-ray detector being positioned relative to each other to define an imaging axis.

### EXAMPLE 14

The biopsy system of any of Examples 1 through 12, the analysis assembly including an x-ray source and an x-ray detector, the x-ray source and the x-ray detector being positioned relative to each other to define an imaging axis, the imaging axis being aligned with a portion of the tissue transport tube.

### EXAMPLES 15

The biopsy system of any of Examples 1 through 14, the sample tray including a cylindrical body defining the plurality of sample chambers, the plurality of sample chambers being arranged in a circular pattern around a central portion of the cylindrical body.

### EXAMPLE 16

An apparatus for use with a biopsy device, the apparatus comprising: a tissue transport tube, the tissue transport tube being configured to receive and transport one or more tissue samples acquired by the biopsy device; a tissue sample holder, the tissue sample holder including a tissue tray, the tissue tray including a plurality of open portions and one or more porous portions associated with one or more open portions of the plurality of open portions, the tissue tray being movable relative to the tissue transport tube to selectively place each open portion or each porous portion in communication with the tissue transport tube; and an analysis assembly defining an imaging axis extending from a detector, a portion of the tissue transport tube being aligned with the imaging axis.

### EXAMPLE 17

The apparatus of Example 16, the one or more porous portions being configured to block a tissue sample relative to a portion of the tissue tray, the one or more porous portions being further configured to permit a flow of vacuum through a portion of the tissue tray.

### EXAMPLE 18

The apparatus of Example 16, the tissue tray further including a plurality of sample chambers, each open portion being in communication with a corresponding sample chamber of the plurality of sample chambers such that each open portion is configured to communicate a tissue sample from the tissue transport tube into the corresponding sample chamber, the one or more porous portions being configured to block a tissue sample relative to a sample chamber of the plurality of sample chambers.

### EXAMPLE 19

The apparatus of any of Examples 16 through 18, the one or more porous portions including a mesh integral with a side portion of the tissue tray.

### EXAMPLE 20

A method of analyzing a tissue sample, the method comprising: transporting a tissue sample acquired by a biopsy device through a tissue transport tube; stopping transportation of the tissue sample through the tissue transport tube proximate a sample tray; analyzing the stopped tissue sample while the stopped tissue sample is disposed within the tissue transport tube; and communicating the tissue sample from the tissue transport tube and into a sample chamber of the tissue tray.

### EXAMPLE 21

The method of Example 20, further comprising rotating the tissue tray to move a receiving opening of the tissue tray into alignment with the tissue transport tube after the step of analyzing the stopped tissue sample.

### EXAMPLE 22

The method of Examples 20 or 21, further comprising rotating the tissue tray to align a sample stopping feature of the tissue tray with the tissue transport tube prior to the step of stopping transportation of the tissue sample.

### EXAMPLE 23

The method of any of Examples 20 through 22, the step of stopping transportation of the tissue sample including stopping transportation of the tissue sample through the tissue transport tube using a portion of the tissue tray.

### Example 24

The method of any of Examples 20 through 23, the step of communicating the tissue sample from the tissue transport tube and into the sample chamber being performed after the step of analyzing the tissue sample.

### EXAMPLE 25

An apparatus for use with a biopsy device, the apparatus comprising: a tissue transport tube, the tissue transport tube being configured to receive and transport one or more tissue samples acquired by the biopsy device; a rotatable tissue tray, the tissue tray defining a plurality of sample chambers and a plurality of receiving openings corresponding to each sample chamber; and a sample stopping assembly positioned proximate the tissue tray, the sample stopping assembly including a sample tube in communication with the tissue transport tube such that the sample tube is configured to communicate the one or more tissue samples from the tissue transport tube and into a sample chamber of the tissue tray, the sample tube including one or more stopping ports extending outwardly relative to a longitudinal axis defined by the sample tube, the one or more stopping ports being configured to receive vacuum to selectively stop the one or more tissue samples within the sample tube for imaging.

### EXAMPLE 26

The apparatus of Example 25, the one or more stopping ports including a porous structure, the porous structure being configured to permit a flow of fluids therethrough while preventing the flow of tissue therethrough.

### EXAMPLE 27

The apparatus of Example 26, the porous structure including a plurality of perforations.

### EXAMPLE 28

The apparatus of Example 27, the plurality of perforations being oriented perpendicularly with respect to the longitudinal axis defined by the sample tube.

### EXAMPLE 29

The apparatus of any of Examples 25 through 28, the one or more stopping ports including a pair of stopping ports, the pair of stopping ports being positioned along a common axis.

### EXAMPLE 30

The apparatus of Example 29, the pair of stopping ports extending perpendicularly relative to the longitudinal axis defined by the sample tube.

### EXAMPLE 31

The apparatus of any of Examples 25 through 30, the sample stopping assembly further including a fluid controller, the fluid controller being in communication with the one or more stopping ports and a proximal end of the sample tube, the fluid controller being configured to selectively divert the flow of fluid from the proximal end of the sample tube to the one or more stopping ports.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A biopsy system, the biopsy system comprising:
(a) a biopsy device, the biopsy device including:
(i) a probe,
(ii) a needle extending from the probe, and
(iii) a cutter, the cutter being movable relative to the needle to sever one or more tissue samples;
(b) tissue handler in communication with the biopsy device, the tissue handler including a tissue sample holder and an analysis assembly, the tissue sample holder including a sample tray, the sample tray including a plurality of sample chambers configured to receive the one or more tissue samples severed by the cutter, the sample tray further including an external surface defining one or more sample stopping features and a plurality of receiving openings, each receiving opening corresponding to a sample chamber; and
(c) a tissue transport tube adapted to connect between the tissue sample holder and the cutter of the biopsy device, the sample tray being configured to move relative to the tissue transport tube to selectively align the one or more sample stopping features or each receiving opening with the tissue transport tube.

2. The biopsy system of claim 1, each sample stopping feature of the one or more sample stopping features being porous.

3. The biopsy system of claim 1, each sample stopping feature of the one or more sample stopping features defining a mesh configuration.

4. The biopsy system of claim 1, the one or more sample stopping features including a single sample stopping feature.

5. The biopsy system of claim 1, the one or more sample stopping features including a single sample stopping feature, the single sample stopping feature being proximate a void defined by the sample tray.

6. The biopsy system of claim 1, the one or more sample stopping features including a plurality of sample stopping features, each sample stopping feature corresponding to a sample chamber of the plurality of sample chambers.

7. The biopsy system of claim 1, the one or more sample stopping features including a plurality of sample stopping features, each sample stopping feature corresponding to a sample chamber of the plurality of sample chambers, each sample stopping feature being disposed proximate a respective receiving opening.

8. The biopsy system of claim 1, the one or more sample stopping features including a plurality of sample stopping features, each sample stopping feature corresponding to a sample chamber of the plurality of sample chambers, each sample stopping feature defining a respective receiving opening.

9. The biopsy system of claim 1, the sample tray being configured to rotate relative to the tissue transport tube.

10. The biopsy system of claim 1, the sample tray being configured to rotate relative to the tissue transport tube about an axis of rotation, each receiving opening and sample stopping feature being aligned along a plane oriented perpendicularly relative to the axis of rotation.

11. The biopsy system of claim 1, the tissue sample holder further including a driver, the driver being configured to rotate the sample tray about an axis.

12. The biopsy system of claim 1, the tissue sample holder further including a driver, the driver being configured to rotate the sample tray about a rotation drive axis, the plurality of sample chambers being aligned along a plane oriented perpendicularly relative to the rotation drive axis.

13. The biopsy system of claim 1, the analysis assembly including an x-ray source and an x-ray detector, the x-ray source and the x-ray detector being positioned relative to each other to define an imaging axis.

14. The biopsy system of claim 1, the analysis assembly including an x-ray source and an x-ray detector, the x-ray source and the x-ray detector being positioned relative to each other to define an imaging axis, the imaging axis being aligned with a portion of the tissue transport tube.

15. The biopsy system of claim 1, the sample tray including a cylindrical body defining the plurality of sample chambers, the plurality of sample chambers being arranged in a circular pattern around a central portion of the cylindrical body.

16. An apparatus for use with a biopsy device, the apparatus comprising:
(a) a tissue transport tube, the tissue transport tube being configured to receive and transport one or more tissue samples acquired by the biopsy device;
(b) a tissue sample holder, the tissue sample holder including a tissue tray, the tissue tray including a plurality of open portions and one or more porous portions associated with one or more open portions of the plurality of open portions, the tissue tray being movable relative to the tissue transport tube to selectively place each open portion or each porous portion in communication with the tissue transport tube; and
(c) an analysis assembly defining an imaging axis extending from a detector, a portion of the tissue transport tube being aligned with the imaging axis.

17. The apparatus of claim 16, the one or more porous portions being configured to block a tissue sample relative to a portion of the tissue tray, the one or more porous portions being further configured to permit a flow of vacuum through a portion of the tissue tray.

18. The apparatus of claim 16, the tissue tray further including a plurality of sample chambers, each open portion being in communication with a corresponding sample chamber of the plurality of sample chambers such that each open portion is configured to communicate a tissue sample from the tissue transport tube into the corresponding sample chamber, the one or more porous portions being configured to block a tissue sample relative to a sample chamber of the plurality of sample chambers.

19. The apparatus of claim 16, the one or more porous portions including a mesh integral with a side portion of the tissue tray.

20. A method of analyzing a tissue sample, the method comprising:
(a) transporting a tissue sample acquired by a biopsy device through a tissue transport tube;
(b) stopping transportation of the tissue sample through the tissue transport tube proximate a sample tray;
(b) analyzing the stopped tissue sample while the stopped tissue sample is disposed within the tissue transport tube; and
(c) communicating the tissue sample from the tissue transport tube and into a sample chamber of the tissue tray.

21-31. (canceled)
